(19) United States
(12) Patent Application Publication
(10) Pub. No.: US 2005/0234561 A1
(43) Pub. Date: Oct. 20, 2005

(54) Surface treatment for implants

(76) Inventors: Michael Nutt, Bartlett, TN (US); Marc Richelsoph, Bartlett, TN (US); Joseph Clift, Bartlett, TN (US)

Correspondence Address: KOHN & ASSOCIATES, PLLC;Suite 410, 30500 Northwestern Highway, Farmington Hills, MI, 48334, US

(21) Appl. No.: 10/828,382

(22) Filed: Apr. 20, 2004

(51) Int. Cl. A61F 2/28; C22C 14/00; C22F 1/18

(52) U.S. Cl. 623/23.53; 606/76; 148/671; 420/417

(57) ABSTRACT

The present invention provides a wear-resistant titanium alloy orthopedic device. Also provided is a method of forming a wear-resistant titanium alloy orthopedic device by deeply diffusing oxygen into the titanium alloy device. A method of hardening a device formed of titanium by deeply diffusing oxygen into the titanium alloy device is provided.

### BACKGROUND OF THE INVENTION

1. Field of the Invention

The present invention relates to wear-resistant metals. More specifically, the present invention relates to wear-resistant titanium for use in orthopedic, namely spinal, implants.

2. Description of Related Art

Orthopedic implants have become increasingly prevalent as millions of patients have been relieved of suffering from joint degenerative diseases and other conditions that affect proper hip, knee, shoulder and other joint function. Total or partial joint replacement procedures involve removal of damaged parts of the relevant joint and replacing them with prosthetic components. During surgery, implant components especially selected to match the patient's needs are implanted in the bones forming the joint. Implant component surfaces disposed against or adjacent to each other in normal operation of the implant are referred to as bearing surfaces. Bearing surfaces can be articulating bearing surfaces, when the two components engage in articulating motion relative to each other, or non-articulating bearing surfaces when they do not engage in articulating motion relative to each other. In a total joint replacement, a bearing component is often interposed between two other components that articulate, or move in a manner that corresponds to the motion of the bones forming the joint. A bearing is frequently used when the articulating components are formed of metal.

The longevity of medical implant devices is of prime importance as it is desirable that the implant should function for the complete lifetime of a patient. This is particularly true if the patient is young. To this end, orthopedic implant materials should preferably combine high strength, corrosion resistance and tissue compatibility. One of the variables affecting the longevity of implants is the rate of wear of the articulating surfaces and long-term effects of metal ion release. Wear debris can contribute to adverse tissue reaction leading to bone resorption, and ultimately the joint must be replaced.

The rate of wear is dependent upon a number of factors which include the relative hardness and surface finish of the materials which constitute the implant, the frictional coefficient between the materials of the implant components, the load applied, and the stresses generated at the articulating surfaces. The most common material combinations currently used in the fabrication of hip-joint implants include femoral heads of cobalt, titanium, or zirconium alloys articulating against acetabular cups lined with organic polymers or composites of such polymers including, for instance, ultra-high molecular weight polyethylene (UHMWPE), femoral heads of polished cobalt alloy in combination with acetabular cups of polished cobalt alloy, and femoral heads of polished alumina in combination with acetabular cups lined with an organic polymer or composite or made of polished alumina.

Of the factors that influence the rate of wear of conventional hip joint implants, the most significant are patient weight and activity level. Additionally, heat generated by friction in the normal use of the implant has been shown to cause accelerated creep and wear of the polyethylene cup. Furthermore, there is a correlation between the frictional moment which transfers torque loading to the cup and the frictional coefficient between the femoral head and the surface of the acetabular cup against which the head articulates. Cup torque has been associated with cup loosening. Thus, in general, the higher the coefficient of friction for a given load, the higher the level of torque generated. Ceramic bearing surfaces have been shown to produce significantly lower levels of frictional torque. Two of the three commonly used hip-joint systems include a metallic femoral head articulating against an ultra high molecular weight polyethylene (UHMWPE) liner inside the acetabular cup. UHMWPE, being a polymeric material, is more susceptible to creep at higher temperatures than the commonly used metal alloys or ceramics due to its dramatically lower melting point (cobalt alloy is 10×, titanium is 12×, and alumina is 14× the melting point of UHMWPE) and is consequently more susceptible to wear than the alloys or ceramics.

The original impetus for the inclusion of polymeric surfaces such as polytetrafluoroethylene (PTFE or Teflon), and eventually UHMWPE, was that they had a lower coefficient of friction than metal surfaces of the time, while some thought the polymer would act sacrificially; they would fail slowly and fail before the harder surface, allowing for an overall extension of the useful life of the device. Additionally, polyethylene was thought to absorb shock much better than harder surfaces, thereby simulating the effect of real cartilage. Considerable research and development has lead to a better understanding of the wear mechanisms, debris morphology, and biological consequences of wear debris, and technologies to improve UHMWPE surfaces. Likewise, similar efforts have lead to technological improvements in processing metals and ceramics that have so improved their wear performance as to permit their use in similar material wear couples (metal-metal or ceramic-ceramic) or in dissimilar wear couples (metal-UHMWPE or ceramic-UHMWPE). One of the more recent innovations utilized oxidized zirconium as a wear surface. While the advance in the art that was realized by the use of oxidized zirconium surfaces articulating against UHMWPE surfaces was a lessening of wear and acetabular cup loosening between the surface of the metallic component and the UHMWPE, the problem was not completely eliminated. Thus, there is still a need for improvements in the wear of implants.

The wear mechanisms include adhesive and abrasive wear, generating a large number of relatively small (micrometer and submicrometer) polyethylene wear particles. The reduction of wear debris generated by orthopedic devices is one of the leading issues regarding long-term performance of orthopedic joint prostheses. Wear debris has been associated with adverse biological responses which can lead to local cell death (osteolysis for bone cells), premature loosening and failure of orthopedic devices, and subsequent need for revision surgery. The majority of wear debris originates from the articulating surface of orthopedic devices, typically a UHMWPE insert or cup surface that is disposed against a metal or ceramic plate or ball surface in a manner such that the surfaces engage in articulating motion relative to each other. (Wear can also occur on non-articulating surfaces, such as a non-mobile bearing component surface against a tibial tray, or the convex, nonarticulating surface of a liner against the inner diameter of an acetabular cup.) Additionally, abrasive third body debris, such as bone cement (for example, polymethylmethacrylate (“PMMA”) bone cement) and bone debris can migrate to the interface between bearing or articulating surfaces, further accelerating abrasive wear due to so-called three body motion.

Researchers have studied various ways to alleviate or eliminate wear and/or abrasion. For example, researchers have attempted to enhance lubrication and alter surface structure of the surfaces that are in sliding or articulating contact. Some investigators have attempted to maintain a constant coefficient of friction between two articulating contacting surfaces by using separately applied lubricants. Such lubricants are commonly solid lubricant films or solid lubricating surfaces. Others have attempted to incorporate a lubricant-type material into the articulating surface itself. The lubrication models do not work or cannot be tolerated because the lubricants introduce undesired contaminants or other undesired physical characteristics into the functionality of the device.

Additionally, various surface structures have been studied. For instance, patterns have been introduced in an effort to enhance the lubrication of the joint and reduce the creation of wear particles. See I. Nishimura, et al. “Modification of the Frictional Surfaces of Artificial Joints,” ASAIO Journal, July-September 1993: M762.

The Nishimura reference discloses that a pattern having a diameter of 0.5 mm and a 1.2 mm pitch showed the maximum reduction in frictional force (25%) and the smallest value for UHMWPE wear (8 μm). When the diameter of the pattern was increased to more than 0.5 mm, the reduction rate decreased and water formation increased. Among other things, this reference fails to discuss the implications that the depth of the pattern has on wear problems, other than fixing the depth at 3 μm on SUS surfaces and 1 mm on UHMWPE surfaces. The reference also discloses a regular pattern of diameter, pitch and depth used on each surface, without taking various geometries of the pattern, anatomical conditions, or various placement patterns into consideration.

There is prior art directed to wear resisting slide members that disclose slide members having circular recessed and projected patterns. The recessed portions are filled with lubricant, and the circular diameter of the recessed portions typically range from 0.2 to 0.8 mm. Circular diameters greater than 0.8 mm cause the area of the projected portions to decrease so that the projected portions cannot support the loads, sliding and lubricating properties are deteriorated due to wear of the slide surface, and unevenness occurs.

Various concepts not considered, however, are that if the recessed portions are not sufficiently deep, synovial fluid can fill the recesses and calcify, essentially re-filling the recesses, thus eliminating the desired benefits. Nor do the currently available references consider the benefits of various positionings of the patterns on the surface or the concept of reducing overall areas of wear.

It has also been determined that metal prostheses are not completely inert in the body. Body fluids act upon the metals causing them to slowly corrode by an ionization process thereby releasing metal ions into the body. Metal ion release from the prosthesis is also related to the articulation and rate of wear of load bearing surfaces because, as can be expected, when a metallic femoral head, for instance, is articulated against UHMWPE, the passive oxide film which forms on the femoral head is constantly removed. The repassivation process constantly releases metal ions during this process. Furthermore, the presence of third-body wear (cement or bone debris) accelerates this process and micro-fretted metal particles can increase friction. Consequently, the UHMWPE liner inside the acetabular cup, against which the femoral head articulates, is subjected to accelerated levels of creep, wear, and torque. A reduction in these deleterious effects also improves the problem of metal ion release.

A number of attempts to correct these problems were the subject of much of the early work in this area. U.S. Pat. No. 4,145,764 to Suzuki taught a metal prosthesis plasma sprayed with a bonding agent that is in turn covered with a porous ceramic coating that would allow the in-growth of bone spicules into the pores. However, the Suzuki patent did not address the issue of friction or wear of orthopedic implant bearing surfaces but confined itself to the single issue of the biocompatibility of metal prostheses and did not address the issue of dimensional changes that occur when applying such a coating.

U.S. Pat. No. 3,677,795 to Bokros discloses the application of a carbide coating over a metallic prosthetic device. The method is intended to produce a prosthetic device which has “excellent compatibility with body tissue and is non-thrombogenic”. However, Bokros does not address the issues of friction, heating, creep and wear of orthopedic implant bearing surfaces, or changes induced in the mechanical properties of the underlying metal due to this high-temperature treatment.

The aforementioned failings of the prior art were addressed in part by Davidson in U.S. Pat. No. 5,037,438. In the '438 patent, Davidson teaches a zirconium or zirconium-containing metal alloy prosthesis coated via in-situ oxidation with a surface of blue-black or black oxidized zirconium that articulates against an organic polymer or polymer-based composite. The oxidized zirconium coating provides the prosthesis with a thin, dense, low friction, wear resistant, biocompatible surface ideally suited for use on articulating surfaces of joint prostheses wherein the surface or surfaces of the joint articulates, translates, or rotates against mating joint surfaces. The oxidized zirconium coating described in the '438 patent can therefore be usefully employed on the femoral heads or inside surfaces of acetabular cups of hip-joint implants or on the articulating surfaces of other types of prostheses, such as knee joints. Notably, the oxidized zirconium coating of the '438 patent was a specific type of oxidized zirconium. Oxidized zirconium presents itself in many forms; among them are white, beige, and blue-black. The white variety is particularly disfavored, as it tends to separate and break off of the substrate. Conventional oxidized zirconium surfaces formed, for example, by simple air oxidation will not be of the blue-black or black variety and will not possess the superior properties of the same that are recited in the '438 patent.

The specific blue-black or black oxidized zirconium coatings of the '438 patent were known in the art of mechanical bearings, having been described in U.S. Pat. No. 2,987,352, which teaches a 700-1100° F. oxidation method to produce the specific blue-black or blue oxidized zirconium coating. A later issuing patent to Haygarth (U.S. Pat. No. 4,671,824) teaches an alternative, salt-bath method to produce the desired coating. The blue-black or black oxidized zirconium of the instant invention possessing the necessary properties is primarily monoclinic crystal structure.

Despite the above, titanium alloys are used extensively in medical implants such as hip joint prostheses, knee joint prostheses, spinal implants, bone plate systems, intramedullary nails, dental implants, cardiovascular implants, ear-nose-and-throat implants, etc., due to their high strength, low modulus, excellent biocompatibility and corrosion resistance. However, a major disadvantage of titanium alloys is their susceptibility to wear and galling. The phenomenon called “galling,” is essentially the sticking together of mating titanium parts which move against each other leading to high friction and wear.

Numerous methods have been proposed for increasing the surface hardness and reducing the wear and galling of titanium alloys. Ceramic coatings, such as titanium nitride, have been deposited on these alloys by processes such as physical vapor deposition and chemical vapor deposition (see, for example, Hintermann, U.S. Pat. No. 4,687,487). However, these ceramic coatings are much harder and stiffer than the base alloy substrate so that there is an abrupt mismatch in the stiffness of the coating and the substrate at the interface between the two. The elastic modulus (stiffness) of a titanium nitride coating is typically about 400 GPa while that of most titanium alloys is about 65 to 130 GPa. This modulus mismatch leads to undesirable stresses at the interface, especially when these components are bent or deformed in any manner, and increases the potential for the coating separating from the substrate by a delamination or spalling mechanism.

Attempts have been made to harden titanium alloys by nitrogen or oxygen ion implantation. In these processes, the titanium alloy substrate is bombarded with nitrogen or oxygen ions using a high voltage apparatus that forces the ions to penetrate the substrate. However, these processes affect the surface to a depth of only about 0.1 micron and peak hardness is not at the surface but slightly below the surface. Hence, the hardened surface tends to wear through relatively quickly.

Titanium alloys have also been hardened by processes such as gas nitriding and salt bath nitriding. These processes also produce a titanium nitride surface on these alloys by penetration of nitrogen into the metal substrate. However, as mentioned above, titanium nitride has a much higher stiffness than the titanium alloy base material, thus being potentially susceptible to detachment from the substrate by delamination or spalling.

There have been a few attempts at oxygen diffusion hardening of titanium alloys, such as for instance disclosed by Streicher, et al., in two conferences (OIMTEC, 1990, Italy, and European Society of Biomechanics, July 8-11, Denmark). However, the alloy used by Streicher, et al., is Ti—6Al—7Nb, which when oxidized would be expected to produce titanium oxide (TiO) or titanium dioxide (TiO₂), both of which have very low shear strength and would be susceptible to detachment.

British Patent No. 1351062 discloses a process for surface hardening a titanium article by heating it in an atmosphere of air, nitrogen, hydrogen or oxygen. However, if heated in air, the surface would be expected to consist of titanium oxide (TiO), titanium dioxide (TiO₂), or titanium nitride with the associated disadvantages described above. If heated in nitrogen, the surface produced would consist of titanium nitride with the associated stiffness mismatch disadvantage described above. If heated in hydrogen, the compound produced would be titanium hydride that is known to severely embrittle and be detrimental to the fatigue strength of titanium alloys. If heated in oxygen, the surface would be expected to consist of titanium oxide (TiO) or titanium dioxide (TiO₂) with the associated low shear strength described above.

In U.S. Pat. No. 5,372,660, Davidson, et al., describes surface hardening Ti—Zr and Ti—Nb—Zr alloy implants by oxygen diffusion at 500° C. (932° F.) in an oxygen-bearing atmosphere of oxygen or water vapor, with a carrier gas of argon or nitrogen, where the oxygen rich layer is less than 50 μm (0.002 inch), and more preferably less than 201 μm (0.0008 inch) thick. Davidson teaches in the same patent that the water vapor is used to obtain dissociated monatomic oxygen but does not reveal how the dissociated hydrogen is prevented from forming the severely embrittling titanium hydride discussed earlier. Davidson also teaches that nitrogen dioxide (NO₂) can be used as one of the oxygen-containing atmospheres for the process, and nitrogen can be used as an inert carrier medium for the oxygen-containing compound, but does not discuss how such nitrogen-bearing atmospheres would avoid formation of titanium nitride and the potential for delamination or spalling discussed earlier. Davidson also teaches the complex oxide surface film in their process (0.1 μm to 10 μm thick) consists of any or all of TiO₂, Ti₂O₃, TiO, and oxides of zirconium and niobium, with the net result that the previously described negative attributes of these titanium oxides are overcome.

Clearly, the development of an effective means for increasing the surface hardness and wear resistance of titanium alloys would be extremely beneficial. In the case of medical implants, abrasive wear may be minimized or eliminated by increasing the hardness of the surface of a titanium alloy. A highly wear resistant titanium implant will not only produce less wear debris that will increase the expected service life of the implant, but will result in reduced levels of metal ion release into the recipient's body tissue. Further, a longer lasting implant may reduce the need for later surgery to replace the implant.

A critical requirement for preventing the failure of wear resistant coatings on a titanium alloy substrate is to provide a relatively thick layer, or case, at the surface of the substrate that provides a transition zone in strength, hardness and stiffness, between the peak properties at the surface and the structural properties at the core. This transition zone reduces or eliminates the mismatch encountered with high hardness, high stiffness coatings deposited on relatively low hardness, low stiffness titanium alloy substrates, and also dramatically improves the support of thin, high hardness, high stiffness diffusion-type coatings applied to relatively low hardness, low stiffness titanium alloy substrates. The thickness of this transition zone, as well as the hardness gradient, permits customizing of the surface and sub-surface properties for the particular wear and loading application.

### SUMMARY OF THE INVENTION

According to the present invention, there is provided a wear-resistant titanium alloy orthopedic device. Also provided is a method of forming a wear-resistant titanium alloy orthopedic device by deeply diffusing oxygen into the titanium alloy device. A method of hardening a device formed of titanium by deeply diffusing oxygen into the titanium alloy device is provided.

### DETAILED DESCRIPTION OF THE INVENTION

Generally, the present invention provides a wear-resistant titanium alloy. The titanium alloy can be used to form orthopedic implants. Alternatively, the titanium alloy can be used to form other articles known to those of skill in the art that require a wear-resistant, non-reactive, titanium surface.

The phrase “wear-resistant” as used herein is intended to be defined as resistant to wear and abrasion. In other words, the surface that is wear-resistant is resilient and thus is impervious to friction related damage.

The term “hardening” or “enhanced hardening” is defined as increased structural hardness of a surface as determined by Vickers hardness or Rockwell C hardness scales. The hardened surface is more wear-resistant and thus unlikely to be damaged during use.

The term “implant” as used herein includes, but is not limited to, orthopedic implants, cardiovascular implants, inner ear implants, dental implants, and other similar implants. Orthopedic implants can include, but are not limited to, spinal implants, hip implants, knee implants, shoulder, elbow, finger, toe, and other joint implants, bone plates, fixation screws, intramedullary nails, compression hip screws, pelvic plates, and other orthopedic implants known to those of skill in the art.

The titanium alloy of the present invention can include biocompatible unalloyed titanium or any titanium alloy known to those of skill in the art to be capable of being formed into an implant. Examples of titanium alloy types include alpha (and near-alpha), alpha-beta, and beta (and near-beta) alloys, preferably alpha-beta or beta (and near-beta) alloys are used in the formation of the implants. Titanium alloys can contain aluminum, tin, molybdenum, zirconium, vanadium, niobium (columbium), tantalum, nickel, copper, chromium, iron, manganese, and silicon, as well as low levels of carbon, oxygen, nitrogen and hydrogen. Alpha alloys do not generally respond to heat treatment, but they are weldable and are commonly used for cryogenic applications, airplane parts, and chemical processing equipment. Alpha-beta alloys can be strengthened by heat treatment and aging, and therefore can undergo manufacturing while the material is still ductile, then undergo heat treatment to strengthen the material, which is a big advantage. The alloys are used in aircraft and aircraft turbine parts, chemical processing equipment, marine hardware, and prosthetic devices. Beta alloys have good hardenability, good cold formability when they are solution-treated, and high strength when they are aged. Beta alloys are slightly denser than other titanium alloys, because of their high alloy content. They are the least creep resistant alloys, they are weldable, and can have yield strengths above 1379 MPa (200 ksi). They are used for highly stressed applications on aircraft.

Specifically, the implant device is preferably made of titanium or a titanium alloy, such as alloys with niobium, tin, zirconium, chromium, aluminum, molybdenum, vanadium, tantalum, silicon, and iron. Some examples of titanium alloys include, but are not limited to, Ti-6Al-4V, Ti-13Nb-13Zr (Davidson & Kovacs 1991), Ti-12Mo-6Zr-2Fe (Wang, Gustavson & Dumbleton 1989), Ti-15Mo, Ti-5Al-2.5Fe, Ti-3Al-2.5V, Ti-3Al-8V-6Cr-4Mo-4Zr, Ti-15Mo-3Al-2.7Nb-0.25Si and Ti-15Mo-2.8Nb-0.2Si (Paris & Bania 1990), Ti-6Al-4V ELI, Ti-6Al-7Nb (Steinemann & Perren 1977), Ti-35Nb-7Zr-5Ta (Ahmed & Rack, 1999), Ti-45Nb, Ti-8V-5Fe-1 Al, Ti-15Mo-5Zr, Ti-11.5Mo-6Zr-4.5Sn, and Ti-5Al-2Sn-4Zr-4Mo-2Cr-1 Fe.

The implant of the present invention can be formed in any manner known to those of skill in the art as being capable of forming a titanium alloy implant. For example, the implant is machined, molded or otherwise shaped.

After the implant has been formed, the surface of the implant is deeply diffused with oxygen. Deep diffusion is defined as diffusing the oxygen-containing substance into the inner layers of the titanium as disclosed herein. In contradistinction with the methods disclosed in the prior art, the oxygen diffusion of the present invention occurs at an elevated temperature for a long enough time that the oxygen is able to deeply diffuse into the titanium alloy. The oxygen in the titanium alloy creates an extremely hard surface on the titanium alloy. The oxygen enriched titanium alloy is wear-resistant and thus is capable of being used as an implant or other device without requiring further surface treatments, such as those described in the prior art. The diffusion of oxygen at the surface produces three distinct regions in the hardened case: a brittle titanium-oxide (TiO₂) top layer, which is subsequently removed; a TiO₂—TiO layer several microns thick; and a hardened region containing dissolved oxygen. Usable hardened titanium having wear resistance is located in the oxygen-rich region and the layer of TiO—TiO₂.

An example of a specific methodology is as follows. An annealed titanium alloy, such as Ti-6Al-4V, is placed in a muffle furnace to develop the highest possible usable surface hardness and ultimate tensile strength (UTS) and thickest practical case (greater than 50 μm or 0.002 inch). A thermal process is chosen that provides an extended aging or annealing cycle (greater than 8 hours), at a temperature above 800° F. (427° C.), preferably above 1200° F. (649° C.), that will not enlarge the grain size of the microstructure, while using an oxygen-containing gas to maximize oxygen surface hardening and avoid hydrogen introduction from the furnace atmosphere and a cooling rate slower than 108° F./hour (60° C./hour.).

An annealing treatment at 1472° F. (800° C.) for 40 hours, in a closed furnace atmosphere of air (assumed 78% N₂, 21% O₂, 1% Ar), followed immediately by a slow cooling rate of 108° F./hour (60° C./hour) provided a maximum surface hardness of 1128 HV (Vickers hardness, 100 gram load) and a usable hardness of 609 HV at 0.0005 inches (12.7 μm) beneath the surface. The hardness declined to a depth of 0.0065 inches (165.1 μm) before reaching the hardness of the core at 318 HV (Vickers hardness, 500 gram load). Depending on the source, the macrohardness of material with a microhardness of 1128 HV should be approximately 72 HRC(C scale Rockwell hardness). Again, depending on the source, the equivalent macrohardness and ultimate tensile strength of material with a microhardness of 609 HV should be approximately 56 HRC and 295 ksi (2034. MPa) respectively. The approximate equivalent macrohardness and ultimate tensile strength of core material with a microhardness of 318 HV should be approximately 32 HRC and 143 ksi (986 MPa) respectively.

Alternative gases, such as carbon dioxide (CO₂), carbon monoxide (CO), oxygen (O₂), ozone (O₃), nitrous oxide (N₂O), or air, alternative gas flow rates, alternative aging or annealing temperatures, alternative aging or annealing times, alternative cooling rates, and alternative titanium alloys, as well as combined thermal cycles, such as solution treatment and age hardening with case hardening, can provide different hardness profiles, different case depths, different process times, and different production costs, depending upon the desired end result.

A reference indicating effect of temperature on oxygen hardening Ti-22V-4Al for 30 minutes in air showed the maximum surface hardness increased with increasing temperature up to 800° C., where hardening reached a plateau. The surface hardness was reported to be approximately 640 HV (50 g load). The same reference indicated the case depth (depth of hardening) increased with increases in temperature from 800° C. to 900° C. For the 800° C., 30 minute treatment, the hardness had dropped to 288 HV (50 g load) at 25 mm (0.98 mils) below the surface, and had dropped to the hardness of the annealed substrate at 200 mm (7.87 mils) below the surface.

Throughout this application, author and year and patents by number reference various publications, including United States patents. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

M. Mushiake et al., Development of Titanium Valve Spring Retainers, SAE Technical Paper Series No. 910428, SAE, 1991, referenced in Materials Properties Handbook: Titanium Alloys, ASM International, 1998, pg. 1154
Oxygen Surface Hardening, Technical Note 9: Descaling and Special Surface Treatments, M. L. Bauccio; Materials Properties Handbook: Titanium Alloys, ASM International, 1998, pg. 1154
Friction and Wear of Titanium Alloys, F. M. Kustas, M. S. Misra, referenced in Materials Properties Handbook: Titanium Alloys, ASM International, 1998, pg. 1169-1176
Contamination During Heat Treatment, Technical Note 6: Heat Treating; Materials Properties Handbook: Titanium Alloys, ASM International, 1998, pg. 1169-1176
I. Nishimura, et al., “Modification of the Frictional Surfaces of Artificial Joints”, ASAIO Journal, July-September 1993: M762
Streicher, R. M., Weber, H., Schon, R., Semlitsch, M., Wear Behavior of Different Ceramic Surfaces in Comparison to TiN and ADH-Treated Ti-6Al-7Nb Alloy Paired with Polyethylene, Paper presented at OIMTEC, Italy, (1990).
Streicher, R. M., Weber, H., Schon, R., Semlitsch, M. F., Tribological Behaviour of a New Wear Couple: ODH Treated TiAlNb-Alloy Against UHMWPE, 7th Meeting of European Soc. of Biomechanics, Denmark, (Jul. 8-11, 1990).

### PATENT DOCUMENTS

U.S. Pat. No. 4,145,764 Suzuki
U.S. Pat. No. 3,677,795 Bokros
U.S. Pat. No. 5,037,438 Davidson
U.S. Pat. No. 2,987,352
U.S. Pat. No. 4,671,824 June 1987 Haygarth
U.S. Pat. No. 4,687,487 August 1987 Hintermann
British Pat. No. 1351062
U.S. Pat. No. 5,372,660 Dec. 13, 1994 Davidson, Mishra, and Poggie
U.S. Pat. No. 5,169,597 Jan. 28, 1991 J. Davidson and P. Kovacs
European Pat. App. 90314051.5 Dec. 20, 1990 J. Davidson and P. Kovacs
U.S. Pat. No. 4,857,269 Aug. 15, 1989 K. Wang, L. Gustavson, & J. Dumbleton
U.S. Pat. No. 4,980,127 Dec. 25, 1990 W. Paris and P. Bania
U.S. Pat. No. 4,040,129 Aug. 9, 1977 S. Steinemann and S. Perren
U.S. Pat. No. 5,871,595 Feb. 16, 1999 T. Ahmed and H. J. Rack
European Pat. Spec. EP 0 707 085 B1 Jul. 1, 1999 T. Ahmed and H. J. Rack

1. A wear-resistant titanium device comprising:
a titanium device;
wear-resistance means for making the titanium device wear-resistant.

2. The device according to claim 1, wherein said device is an orthopedic device.

3. The device according to claim 2, wherein said orthopedic device is selected from the group consisting essentially of orthopedic implants, inner ear implants, dental implants and cardiovascular implants.

4. The device according to claim 3, wherein said orthopedic implants are selected from the group consisting essentially of spinal implants, hip implants, knee implants, shoulder, elbow, finger, toe, other joint implants, bone plates, fixation screws, intramedullary nails, compression hip screws, pelvic plates, and other orthopedic implants.

5. The device according to claim 1, wherein said titanium alloy is formed of titanium and a metal selected from the group consisting essentially of niobium, tin, zirconium, chromium, aluminum, molybdenum, vanadium, tantalum, silicon, and iron.

6. The device according to claim 5, wherein said titanium alloy is selected from the group consisting essentially of Ti-6Al-4V, Ti-13Nb-13Zr, Ti-12Mo-6Zr-2Fe, Ti-5Al-2.5Fe, Ti-3Al-2.5V, Ti-3Al-8V-6Cr-4Mo-4Zr, Ti-15Mo-3Al-2.7Nb-0.25Si, Ti-15Mo-2.8Nb-0.2Si Ti-6Al-4V ELI, Ti-6Al-7Nb, Ti-35Nb-7Zr-5Ta, Ti-45Nb, Ti-18V-5Fe-1Al, Ti-15Mo-5Zr, Ti-11.5Mo-6Zr-4.5Sn, Ti-5Al-2Sn-4Zr-4Mo-2Cr-1 Fe, and Ti-15Mo.

7. A wear-resistant titanium alloy orthopedic device, wherein said device has a core hardness of at least 28 HRC and a substantially increased near surface hardness at least 0.0005 inches beneath a surface of the device.

8. The device according to claim 7, wherein said near surface hardness is at least 50 HRC.

9. The device according to claim 7, wherein said near surface hardness is increased at least 0.0065 inches beneath the surface.

10. The device according to claim 7, wherein said device decreases in hardness from the surface to the core.

11. The device according to claim 7, wherein said titanium alloy is formed of titanium and a metal selected from the group consisting essentially of niobium, tin, zirconium, chromium, aluminum, molybdenum, vanadium, tantalum, silicon, and iron.

12. The device according to claim 11, wherein said titanium alloy is selected from the group consisting essentially of Ti-6Al-4V, Ti-13Nb-13Zr, Ti-12Mo-6Zr-2Fe, Ti-5Al-2.5Fe, Ti-3Al-2.5V, Ti-3Al-8V-6Cr-4Mo-4Zr, Ti-15Mo-3Al-2.7Nb-0.25Si, Ti-15Mo-2.8Nb-0.2Si, Ti-6Al-4V ELI, Ti-6Al-7Nb, Ti-35Nb-7Zr-5Ta, Ti-45Nb, Ti-8V-5Fe-1 Al, Ti-15Mo-5Zr, Ti-11.5Mo-6Zr-4.5Sn, Ti-5Al-2Sn-4Zr-4Mo-2Cr-1 Fe, and Ti-15Mo.

13. The device according to claim 7, wherein said orthopedic device is selected from the group consisting essentially of orthopedic implants, inner ear implants, dental implants and cardiovascular implants.

14. The device according to claim 13, wherein said orthopedic implants are selected from the group consisting essentially of spinal implants, hip implants, knee implants, shoulder, elbow, finger, toe, other joint implants, bone plates, fixation screws, intramedullary nails, compression hip screws, pelvic plates, and other orthopedic implants.

15. A wear-resistant titanium alloy spinal implant.

16. The device according to claim 15, wherein said titanium alloy is formed of titanium and a metal selected from the group consisting essentially of niobium, tin, zirconium, chromium, aluminum, molybdenum, vanadium, tantalum, silicon, and iron.

17. The device according to claim 16, wherein said titanium alloy is selected from the group consisting essentially of Ti-6Al-4V, Ti-13Nb-13Zr, Ti-12Mo-6Zr-2Fe, Ti-5Al-2.5Fe, Ti-3Al-2.5V, Ti-3Al-8V-6Cr-4Mo-4Zr, Ti-15Mo-3Al-2.7Nb-0.25Si, Ti-15Mo-2.8Nb-0.2Si, Ti-6Al-4V ELI, Ti-6Al-7Nb, Ti-35Nb-7Zr-5Ta, Ti-45Nb, Ti-8V-5Fe-1 Al, Ti-15Mo-5Zr Ti-11.5Mo-6Zr-4.5Sn, Ti-5Al-2Sn-4Zr-4Mo-2Cr-1Fe, and Ti-15Mo.

18. A wear-resistant titanium alloy orthopedic device.

19. The device according to claim 18, wherein said titanium alloy is formed of titanium and a metal selected from the group consisting essentially of niobium, tin, zirconium, chromium, aluminum, molybdenum, vanadium, tantalum, silicon, and iron.

20. The device according to claim 19, wherein said titanium alloy is selected from the group consisting essentially of Ti-6Al-4V, Ti-13Nb-13Zr, Ti-12Mo-6Zr-2Fe, Ti-5Al-2.5Fe, Ti-3Al-2.5V, Ti-3Al-8V-6Cr-4Mo-4Zr, Ti-15Mo-3Al-2.7Nb-0.25Si, Ti-15Mo-2.8Nb-0.2Si, Ti-6Al-4V ELI, Ti-6Al-7Nb, Ti-35Nb-7Zr-5Ta, Ti-45Nb, Ti-8V-5Fe-1Al, Ti-15Mo-5Zr, Ti-11.5Mo-6Zr-4.5Sn, Ti-5Al-2Sn-4Zr-4Mo-2Cr-1 Fe, and Ti-15Mo.

21. The device according to claim 18, wherein said orthopedic device is selected from the group consisting essentially of orthopedic implants, inner ear implants, dental implants and cardiovascular implants.

22. The device according to claim 21, wherein said orthopedic implants are selected from the group consisting essentially of spinal implants, hip implants, knee implants, shoulder, elbow, finger, toe, other joint implants, bone plates, fixation screws, intramedullary nails, compression hip screws, pelvic plates, and other orthopedic implants.

23. A method of forming a wear-resistant titanium alloy orthopedic device by deeply diffusing oxygen into the titanium alloy device.

24. The method according to claim 23, wherein said diffusing step includes heating the titanium device in the presence of an oxygen containing substance, thereby deeply diffusing oxygen into the titanium device.

25. The method according to claim 24, wherein said heating step includes heating the titanium device to a temperature of at least 800° F.

26. The method according to claim 24, wherein said diffusing step includes heating the titanium device in a substance selected from the group consisting essentially of air, carbon dioxide, carbon monoxide, oxygen, ozone, and nitrous oxide.

27. A method of hardening a device formed of titanium by deeply diffusing oxygen into the titanium alloy device.

28. The method according to claim 27, wherein said diffusing step includes heating the titanium device in the presence of an oxygen containing substance, thereby deeply diffusing oxygen into the titanium device.

29. The method according to claim 28, wherein said heating step includes heating the titanium device to a temperature of at least 800° F.

30. The method according to claim 27, wherein said diffusing step includes heating the titanium device in a substance selected from the group consisting essentially of air, carbon dioxide, carbon monoxide, oxygen, ozone, and nitrous oxide.

31. A method of enhancing wear properties of titanium alloy devices by deeply diffusing oxygen into the titanium alloy device.

32. The method according to claim 31, wherein said diffusing step includes heating the titanium device in the presence of an oxygen containing substance, thereby deeply diffusing oxygen into the titanium device.

33. The method according to claim 32, wherein said heating step includes heating the titanium device to a temperature of at least 800° F.

34. The method according to claim 31, wherein said diffusing step includes heating the titanium device in a substance selected from the group consisting essentially of air, carbon dioxide, carbon monoxide, oxygen, ozone, and nitrous oxide.
